(19) United States
(12) Patent Application Publication
(10) Pub. No.: US 2023/0364603 A1
(43) Pub. Date: Nov. 16, 2023

(54) LATERAL FLOW IMMUNOASSAY CASING AND COLLECTION DEVICE

(71) Applicants: Mayo Foundation for Medical Education and Research, Rochester, MN (US); Safe Health Systems, Inc., Los Angeles, CA (US)

(72) Inventors: Ken Mayer, Los Angeles, CA (US); Steven J. Lester, Scotsdale, AZ (US)

(21) Appl. No.: 18/013,674

(22) PCT Filed: Jul. 1, 2021
(86) PCT No.: PCT/US2021/040120

§ 371 Date: Dec. 29, 2022

Related U.S. Application Data
(60) Provisional application No. 63/047,801, filed on Jul. 2, 2020.

(51) Int. Cl. B01L 3/00; G01N 33/543

(52) U.S. Cl. CPC B01L 3/5023; G01N 33/54388; B01L 2200/16; B01L 2300/0825; B01L 2200/0689; B01L 2300/043; B01L 2300/0672

(57) ABSTRACT

The subject matter disclosed herein includes a cassette device for lateral flow immunoassay testing, including a housing; a receptacle located on a top portion of the housing; and a gating device. The housing defines an interior space to accommodate a test strip. The receptacle is arranged to hold a fluid above the test strip. The gating device includes a sealing portion that selectively seals an opening defined in a base of the receptacle. The gating device is configured to be actuated between (i) a first position in which the sealing portion engages the opening in the base of the receptacle to form a seal and (ii) a second position in which the sealing portion disengages the opening in the base of the receptacle to break the seal.

### CROSS-REFERENCE TO RELATED APPLICATIONS

This application claims the benefit of priority to U.S. Provisional Application Ser. No. 63/047,801, filed Jul. 2, 2020. The disclosure of the prior application is considered part of (and is incorporated by reference in) the disclosure of this application.

### FIELD OF THE DISCLOSURE

The disclosure relates to immunoassays and more specifically, to a single use or self-administered lateral flow immunoassay cassette with sample receptacle.

### BACKGROUND

Test strips and cassettes have been produced for consumer home-use to facilitate testing of self-collected bodily material samples (specimens). Collected samples are generally mixed with a reagent solution in a separate container and subsequently applied to an application location on a test strip or cassette. After waiting for the sample to migrate to and react with all reagents present in one or more analytical areas, the test result can be determined by the user, according to instructions provided by the test supplier.

One method of determining test result involves visually inspecting for the presence or absence of areas, markers, or symbols on the surface of the strip or test housing. The presence or absence of signal can then be interpreted according to provided instructions. For tests where a semi-quantitative result is sought, methods can include compare the test result against a visual comparison sheet which provides a number of example signals, or to a set of standards printed on the strip or test itself. The user can then determine the “best match” against the comparison sheet provided by the test supplier. The comparison could also be done using mobile electronic device with a visual analysis algorithm. These mobile devices and algorithms are not part of this disclosure. A growing range of such self-administered tests is available for conditions such as pregnancy, bacterial infections, viral infections, cancers, cholesterol, antibodies, and others.

### SUMMARY

Disclosed herein is an apparatus for housing lateral flow immunoassay (LFIA) paper-based tests, and methods of use of the same. In some embodiments, the apparatus enables a more simplified, fault tolerant method for biological specimen collection and preparation by professional and non-professional users. The apparatus design can reduce the number of steps required for sample preparation through provision of a reservoir component in the test cassette that contains the reagent solution that is to be mixed with the sample. The design allows for the collected sample to be contacted with a reagent solution without the need for an external container or manual reagent mixing by the user. The sample is permitted to react with the reagent solution for a defined period of time and then flowed out of the receptacle and absorbed by the LFIA paper test strip housed in the test cassette device. Some embodiments can make the process more user friendly, less prone to human error, improve test validity, improve result quality, and improve user safety.

In one aspect, the apparatus disclosed herein can be a cassette device for lateral flow immunoassay testing, including a housing; a receptacle located on a top portion of the housing; and a gating device. The housing can define an interior space to accommodate a test strip. The receptacle can be arranged to hold a fluid above the test strip. The gating device can include a sealing portion that selectively seals an opening defined in a base of the receptacle. The gating device can be configured to be actuated between (i) a first position in which the sealing portion engages the opening in the base of the receptacle to form a seal and (ii) a second position in which the sealing portion disengages the opening in the base of the receptacle to break the seal.

Embodiments of the cassette device for lateral flow immunoassay testing can include one or more of the following features. When the gating device can be in the first position, the seal that can be formed between the sealing portion of the gating device and the opening in the base of the receptacle can be operable to substantially prevent fluid from flowing out of the receptacle through the opening.

When the gating device is in the second position and when the fluid is present in the receptacle, the fluid can be capable of flowing through the opening in the base of the receptacle onto a portion of the test strip positioned beneath the receptacle. The receptacle and the test strip can be positioned with respect to each other such that the fluid flows through the opening onto a sample portion of the test strip.

The sealing portion of the gating device can include a post having an outer diameter that corresponds to an inner diameter that enables the post to be slidably received through the opening in the base of the receptacle.

The housing can further include a second opening located a positive distance from the receptacle, and the gating device can further include a button portion that protrudes through the second opening such that, when the gating device can be in the first position, the button portion can be capable of being depressed by an external force to actuate the gating device into the second position.

The housing can include a viewing window that can include an opening positioned above one or more reporting areas of the test strip.

The gating device can be biased by default in the first position. The gating device can further include a spring located in the interior space of the housing, wherein the spring can be operable to facilitate biasing of the gating device in the first position.

The fluid can be a mixture of a reagent and a biological sample collected from a test subject. The biological sample can include blood, serum, saliva, or urine.

The cassette device can be configured for use in testing for COVID-19.

The cassette device can be configured for use in testing for a bacterial infection, a viral infection, a cancer, a cholesterol level, or an antibody.

The cassette device can further include a fluid reservoir located on the top portion of the housing adjacent to the receptacle; and a channel between the fluid reservoir and the receptacle. The fluid reservoir can be arranged to hold a solution for mixing with a biological sample disposed in the receptacle. The cassette device can further include an actuator that, when actuated, causes a seal to break that (i) opens the channel to permit the solution to flow from the fluid reservoir to the receptacle or (ii) punctures a sack of the fluid reservoir that holds the solution to permit the solution to flow out of the sack and through the channel into the receptacle. The actuator can include a hinged lid having one or more protrusions.

Other advantages will be apparent from the description, the drawings, and the claims.

### BRIEF DESCRIPTION OF THE DRAWINGS

FIG. 1 is a schematic diagram of a lateral flow immunoassay (LFIA) testing strip.

FIG. 2A is a schematic diagram of a sample containing target analytes contacting a lateral flow immunoassay testing strip.

FIG. 2B is a schematic diagram of a sample containing target analytes flowing across a lateral flow immunoassay testing strip.

FIG. 2C is a schematic diagram of a lateral flow immunoassay testing strip after the sample containing target analytes has completed reacting with the test areas.

FIGS. 3A-3I depict various views of a cassette device for lateral flow immunoassay testing.

In the figures, like symbols indicate like elements.

### DETAILED DESCRIPTION

Disclosed herein is an apparatus for housing a lateral flow immunoassay (LFIA) paper-based test that enables self-administration of a sample and buffer solution.

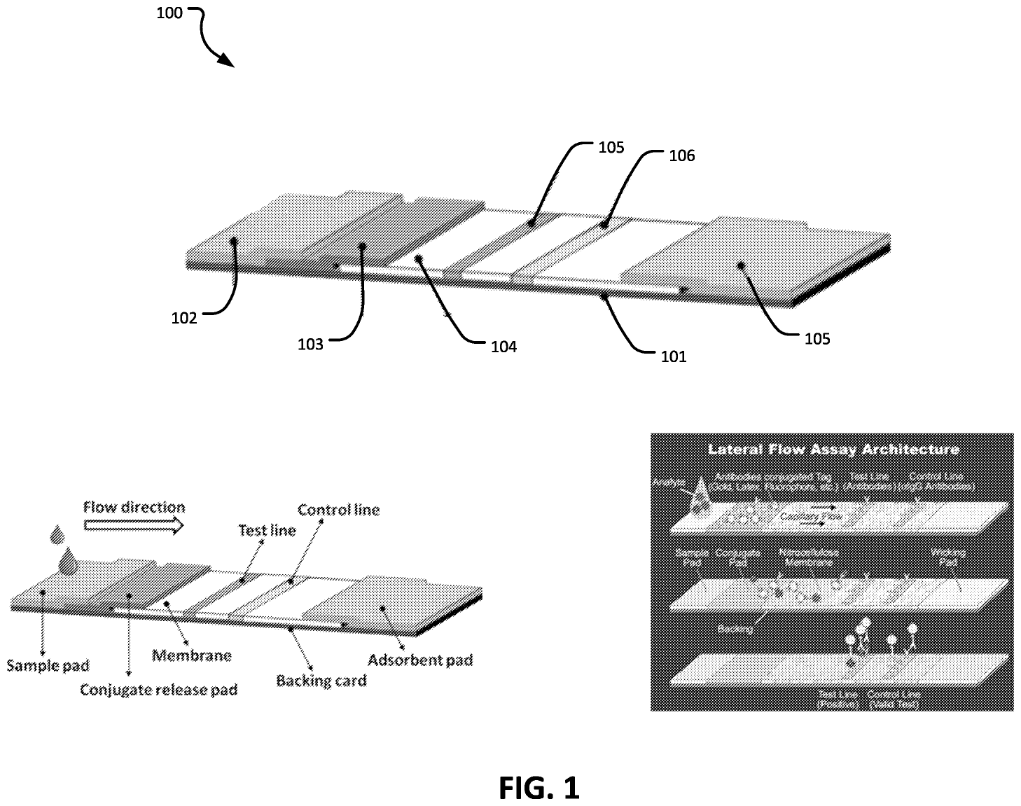

FIG. 1 depicts an example LFIA test strip 100 used for testing for the presence or absence of target analytes within a sample. The LFIA test strip 100 can have multiple components affixed to the top surface of a backing element 101 (e.g., a substrate) that provides structural support to the test strip. Non-limiting examples of the backing 101 include non-absorbent materials such as polymers, metals, or glasses. The components of the LFIA test strip 100 affixed to the backing 100 are arranged to transport a sample containing analytes through one or more reporting areas.

FIG. 1 depicts the LFIA test strip 100 with multiple components consisting of a sample pad 102, a conjugate pad 103, a capillary membrane 104, and an absorbent pad 105. The sample pad 102 can be an absorbent material where sample containing solutions are applied. The sample pad 102 abuts the conjugate pad 103, which stores one or more conjugating molecules. The conjugate pad 103 abuts the capillary membrane 104 responsible for the transportation of the sample containing solution across one or more reporting areas. The terminal absorbent pad 105 can abut the capillary membrane to facilitate sample wicking through the capillary membrane 104 and provide a reservoir in which to store sample containing solution after transport through the capillary membrane 104.

In general, the capillary membrane 104 of the LFIA test strip 100 can have one or more reporting areas for reporting the presence of absence of a target analyte. For example, the target analyte can be an antibody, or antigen. For example, the reporting areas can show a visual signal in the presence or absence of the target analyte or control conjugate. This visual signal can include, but is not limited to, a color change, or the appearance of a shape, mark or symbol. For example, FIG. 1 depicts the LFIA test strip 100 as having two reporting areas in the shape of a line: a testing area 105 and a control area 106.

The capillary membrane 104 of the LFIA test strip 100 can have one or more separated channels for sample wicking. In some embodiments, the capillary membrane 104 can be divided into one or more disconnected sections aligned between the conjugate pad 103 and the absorbent pad 105. This can allow sample from the sample pad 102 to flow through the conjugate pad 103 and into the one or more capillary membrane 104 sections. Each capillary membrane 104 section can have one or more reporting areas corresponding to a different testing 105 or control 106 area.

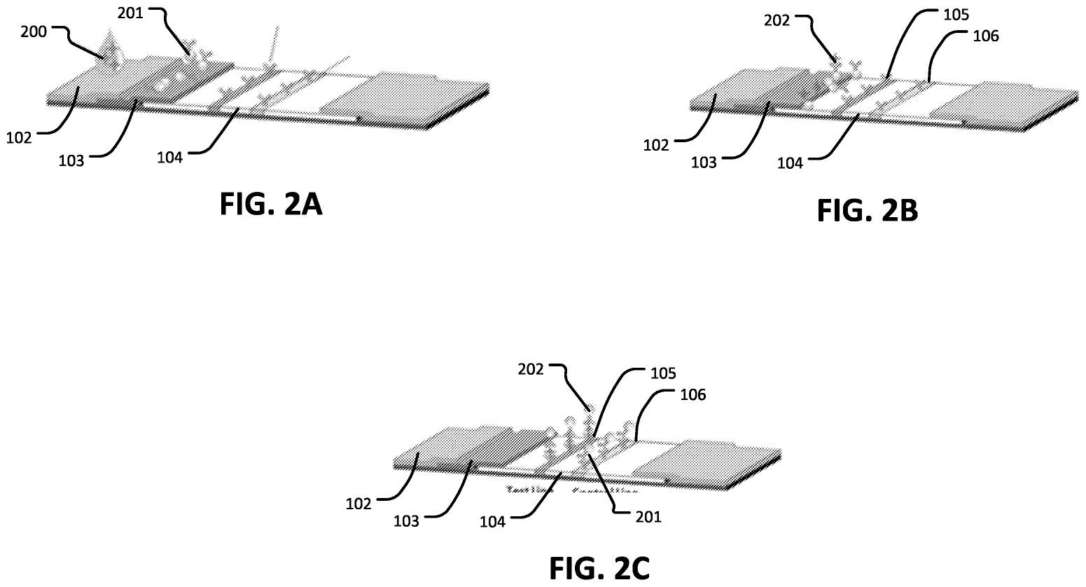

Referring now to the images of FIG. 2, an exemplary testing outline for the use of the LFIA test strip 100 is depicted. In FIG. 2A, a sample 200 containing a target analyte is shown being applied to the sample pad 102 of the LFIA test strip 100. The sample pad 102 can act as a sponge and can hold an excess of sample. Once the sample 200 has soaked the sample pad 102, the fluid can flow to the conjugate pad 103 in which one or more conjugates 201 are stored. The conjugate 201 can be an analyte binding agent (e.g., antibody) that has been specifically affixed with a reporter molecule. For example, the reporter molecule can be a molecule capable of providing a visual signal at high concentrations (e.g., colloidal gold). Further, the analyte binding agent of the conjugate 201 can be capable of forming a specific complex with the target analyte.

The conjugate pad 103 can further contain one or more reagents that enable a chemical reaction between the target analyte and the conjugate 201. As depicted in FIG. 2B, as the sample 200 containing the target analyte flows from the sample pad 102 to the conjugate pad 103, the conjugate 201 can specifically bind with the target analyte to form a complex 202. The conjugate pad 201 may contain more conjugate 201 than is necessary to bind the entire population of target analyte. Alternatively, the reaction between the conjugate 201 and the target analyte may not be efficient. For example, the entire population of target analyte may not bind to a conjugate 201 to form a complex 202 leaving excess target analyte and conjugate 201 in the sample solution. As the sample 200 flows through the conjugate pad 103, complex 202, excess target analyte, and unbound conjugate 201 can flow into the capillary membrane 104.

As described above, the capillary membrane can have one or more reporting areas. The reporting areas can contain additional analyte binding agent (e.g., antibody) that is immobilized within the one or more reporting areas of the capillary membrane 104. For example, FIG. 2B depicts the testing area 105 and control area 106 of FIG. 1. The immobilized analyte binding agents of the testing area 105 can be capable of specifically binding the target analyte. As the sample 200 containing complex 202, excess target analyte, and unbound conjugate 201 flows through the testing area 105, the target analyte of the complex 202 and excess target analyte can be specifically bound by the immobilized analyte binding agents. This can cause the complex 202 to become spatially concentrated. The increased spatial concentration of the reporter molecule in the complex 202 can cause a signal when the complex becomes immobilized in the testing area 105. The signal can be an indication of the presence of target analyte within the testing area.

In some embodiments, the positions of the reporting areas can be randomized. In some embodiments, the reporting areas can be an array of spots, lines, or other shapes. In some embodiments, the reporting areas can include more than one analyte binding agent. In some embodiments, the reporting areas can include more than one concentration of analyte binding agents. In some embodiments, the reporting areas can include permanent or temporary inks.

The sample 200 containing unbound conjugate 201 can continue to flow through the capillary membrane 104 to the control area 106 of FIG. 2B. The control area 106 can have a means of immobilizing the unbound conjugate 201 within the control area 106. For example, a population of target analyte can be immobilized within the control area with which the conjugate 201 can form a complex 202. For a further example, a target analyte competitor that is also capable of forming a complex 202 with the analyte binding agent of the conjugate 201 can be immobilized within the control area 106. The unbound conjugate 201 can become specifically bound in the control area 106. This can cause the unbound conjugate 201 to become spatially concentrated. The increased spatial concentration of reporter molecule in the control area 105 can cause a signal.

FIG. 2C shows how the result of the LFIA test strip 100 can be determined by the presence or absence of signal in the testing area 105, the control area 106, or both. FIG. 2C depicts bound complex 202 in the testing area 105 and bound conjugate in the control area 106. A signal in both the testing area 105 and the control area 106 may indicate the positive presence of the target analyte. A signal in the control area 106 and no signal in the testing area 105 can indicate a valid assay with a negative presence of the target analyte. An indeterminate signal in either the testing area 105 or control area 106 may indicate an inconclusive test (e.g., failed test).

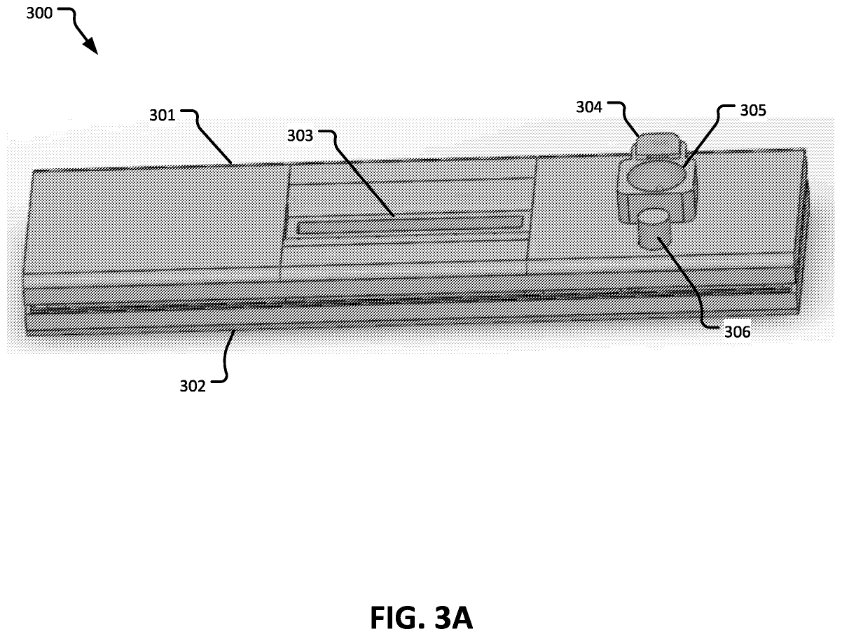

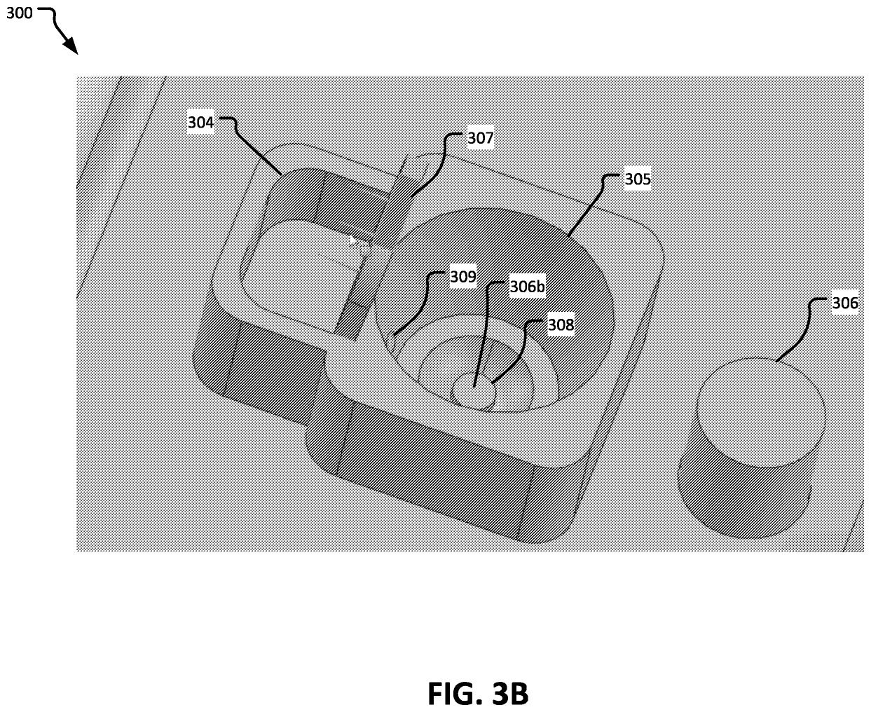

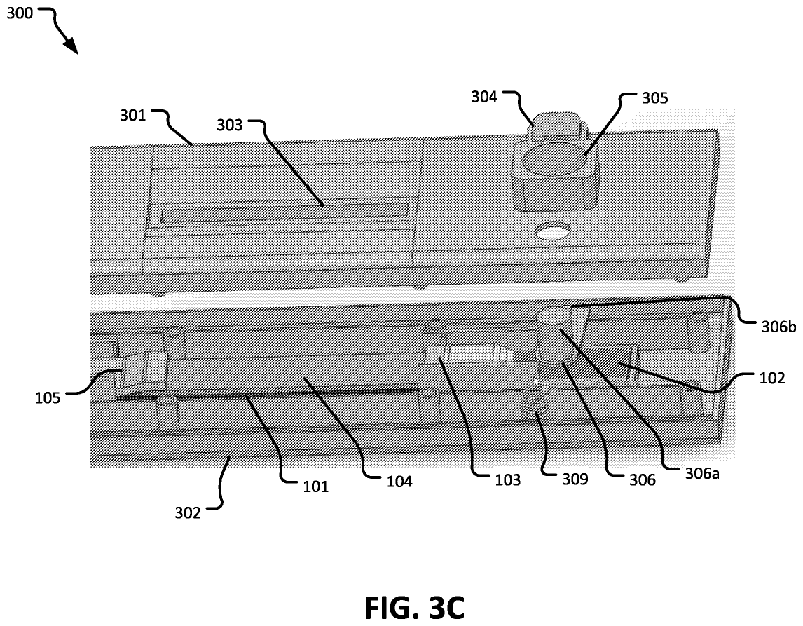

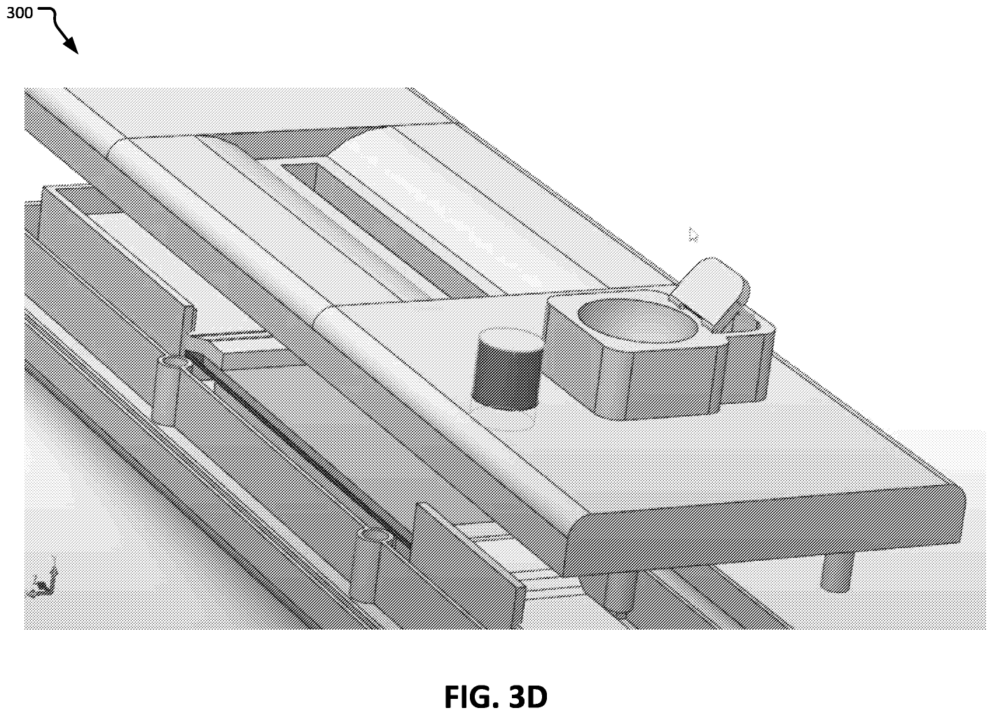

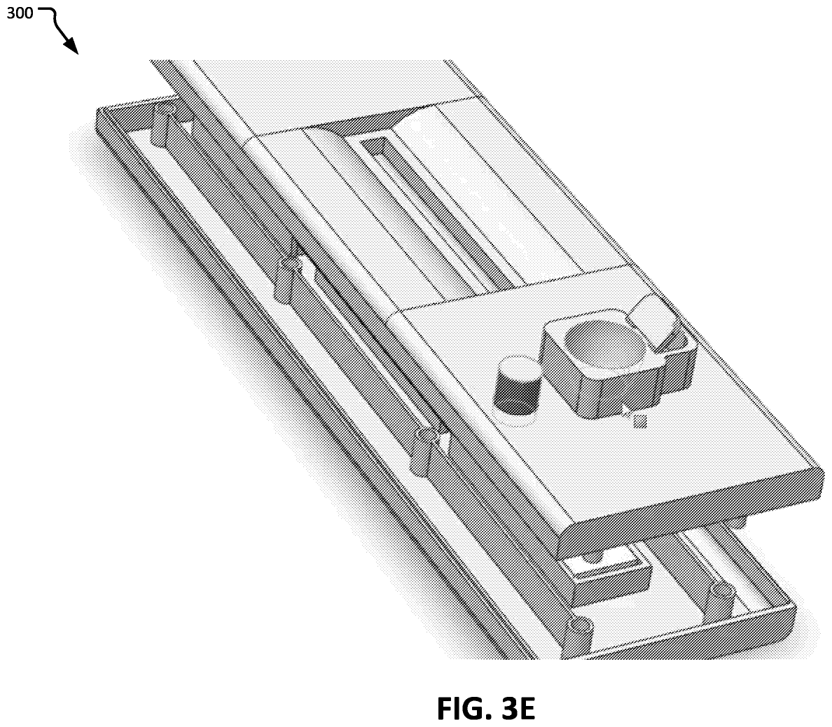

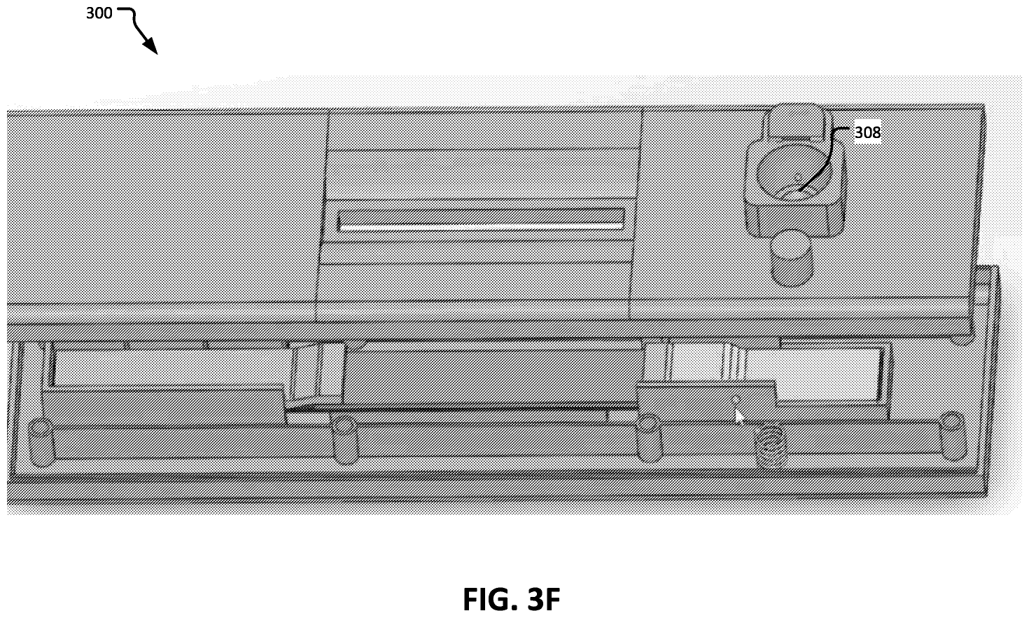

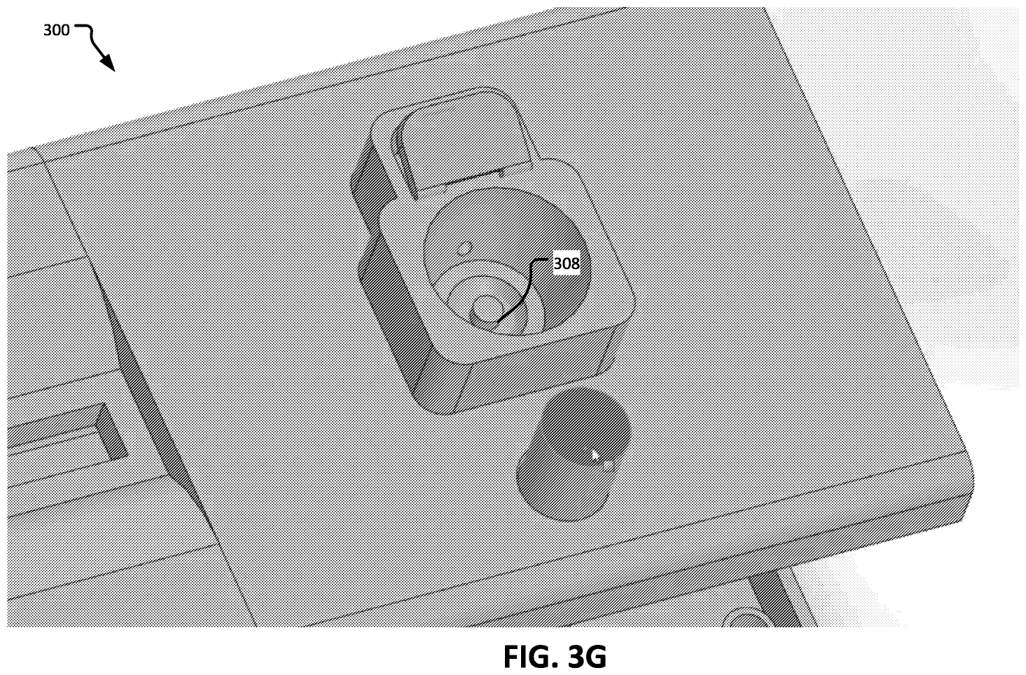

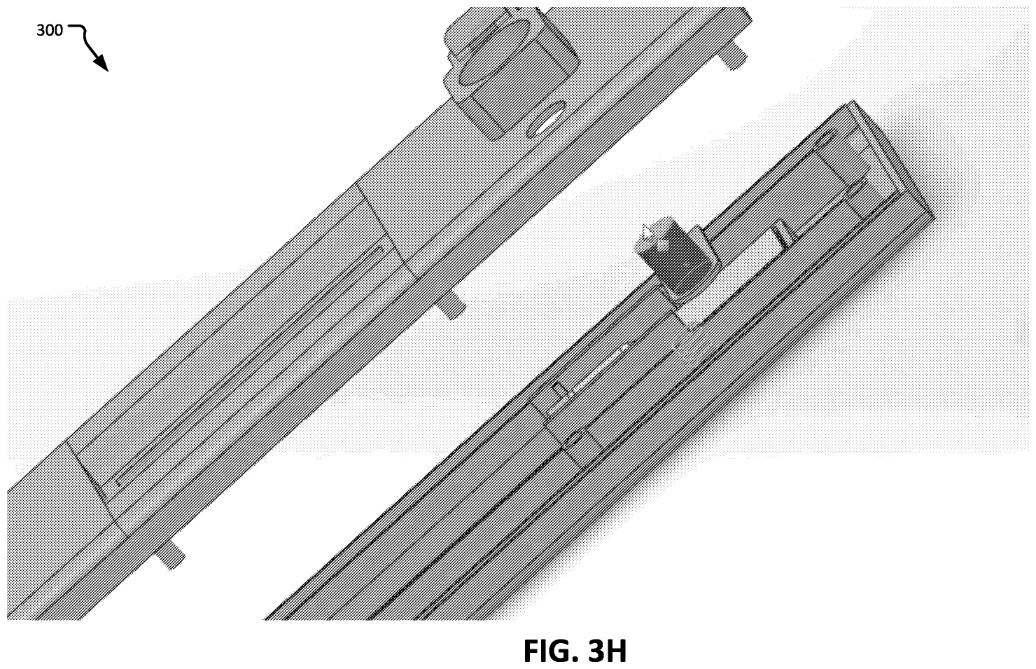

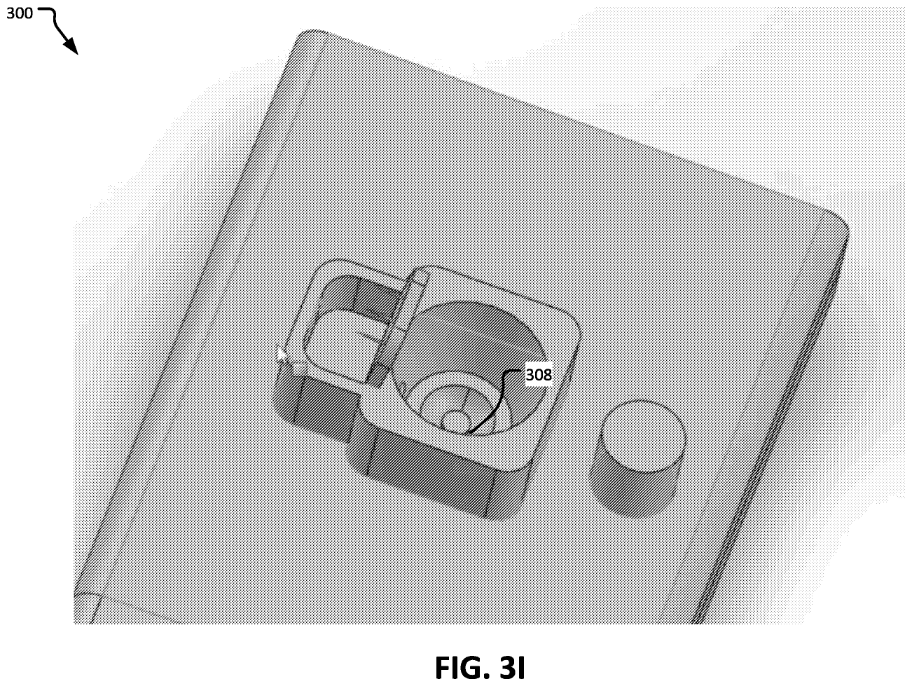

The LFIA test strip 100 can be disposed in a cassette 300, for which various views are depicted in the images of FIG. 3. The cassette 300 can fully or partially house the LFIA test strip 100. The cassette 300 maintains the mechanical and biological integrity of the LFIA test strip 100 housed therein. For example, if the cassette 300 fully encloses the LFIA test strip 100, the cassette 300 can maintain the relative positioning of the components of the LFIA test strip 100 without need for mechanical or chemical (e.g., adhesive) fixation that may interfere with proper conduction of the assay. In general, the cassette 300 can be constructed of a material suitable for the containment of the biological and chemical components necessary for the LFIA test strip 100 to be run. Non-limiting examples of these materials include polymers, metals, or glasses.

The cassette 300 includes an upper housing 301 and a lower housing 302. For example, FIG. 3A shows the upper housing 301 with a viewing window 303. In general, the cassette 300 can have one or more viewing windows 303 for the purpose of providing the user with a view to the reporting areas of the LFIA test strip 100. The viewing window can be of any shape or size that allows for viewing of the reporting areas. The viewing window can optionally be of a shape or size that allows for the viewing of additional components of the LFIA test strip 100.

To allow for integrated sample preparation, buffer mixing, and variable testing durations, the cassette 300 includes a sample preparation area including a buffer reservoir 304, a sample receptacle 305, and a gating device 306. In general, the sample preparation area can include one or more buffer reservoirs 304, sample receptacle 305, gating device 306, or a combination of these. The sample preparation area is configured to be located above the sample pad 102 of the LFIA test strip 100. The positioning of the sample preparation area is further described in FIG. 3C.

FIG. 3B depicts a close up view of the sample preparation area including the buffer reservoir 304, sample receptacle 305, and gating device 306 of the cassette 300. This view shows the buffer reservoir 304, sample receptacle 305, and gating device 306 proximally arranged within the sample preparation area and the buffer reservoir 304

In general, the buffer reservoir 304 can contain one or more reagent solutions (e.g., buffer) for the LFIA test strip 100 in one or more discrete sealed chambers (e.g., one or more, two or more, or three or more). The solution(s) within the one or more sealed chambers can be released upon actuation of a lid 307 by the user. For example, the cassette 300 can include a hinged lid 307 having one or more needles or other pointed protrusions on its underside such that when the lid 307 is folded down, the needles/protrusions puncture a sack containing the buffer solution(s) in the reservoir 304. The hinged lid 307 in the example of FIG. 3B may be depressed by the user and the needles driven into the one or more sealed chambers thereby opening (e.g., puncturing) them and allowing their contents to mix.

In general, the method of opening the one or more sealed chambers of the buffer reservoir 304 can include any method that may be actuated by the user. For example, the opening of the sealed chambers can occur by addition of the sample specimen to the sample receptacle 305. When the sample is delivered via swab, the placement of the swab within the sample receptacle 305 may actuate the opening of the sealed chambers. As a further example, the sealed chambers of the buffer reservoir can be sealed by a removable strip. The user can remove the gate thereby opening the sealed chambers and allowing the contents of the sealed chambers to mix.

The sample receptacle 305 can include an aperture and a depression that defines a hole/opening 308 at the base of the receptacle 305. FIG. 3B shows a blocking post 306b protruding through the hole/opening 308, which is described further with respect to FIG. 3C. Additional views of the hole/opening 308 can be seen in FIGS. 3F, 3G, and 3I. In general, the aperture and depression can be formed in any suitable shape capable of receiving and holding the biological sample/specimen and allowing collection of mixed buffer solution (e.g., reagent mixed with the sample/specimen). Non-limiting examples of the shapes can be circular, cylindrical, or hemispherical. In general, the sample can be deposited in the sample receptacle 305. Non-limiting examples of samples can include biological liquids such as blood, plasma, saliva, or urine. Non-limiting examples of sample delivery methods to the sample receptacle 305 can be via swab, dropper, syringe, pipette, or direct placement of the sample.

In general, the sample receptacle 305 can be constructed to match the means of sample delivery. For example, the sample receptacle 305 can be constructed to include a locking mechanism such that a syringe with a locking mechanism (e.g., a luer lock) can be locked into the sample receptacle before delivering the collected specimen. In the example of collected specimen being delivered by swab, the sample receptacle 305 can be of a shape and size (e.g., hemispherical) to conform to the swab used for sample delivery.

A small channel 309 connects the buffer reservoir 304 to the sample receptacle 305. The channel 309 extends from a first opening located at or near a base of the buffer reservoir 304 to a second opening located in a sidewall of the sample receptacle 305. The first opening in the buffer reservoir 304 is elevated above the second opening in the sample receptacle 305 to permit flow of reagent from the buffer reservoir 304 to the sample receptacle 305. Upon user actuation of a mechanism to release the reagent in buffer reservoir 304 (and any mixing of solutions if multiple chambers are present), the reagent flows through the channel 309 into the sample receptacle 305 where it comes into contact with the sample specimen. Contacting the sample with the reagent solutions creates a mixed reagent-sample solution. The user allows the reagent to mix with the sample for a defined period of time (i.e., the mixing interval). For example, the mixing interval can range from 1 s or more (e.g., 1 s or more, 10 s or more, 30 s or more, 60 s or more). Depending on the nature of the test and the specific reagents and samples involved, the mixing time interval can include a non-limiting range from 1 m or more (e.g., 1 m or more, 10 m or more, 30 m or more, 60 m or more).

In some embodiments, the sample receptacle 305 and sample pad 102 are positioned along the longitudinal axis of the capillary membrane 104 (e.g., a “T shape”). In such embodiments, the mixed reagent-sample solution from the sample receptacle 305 can flow onto the sample pad 102 and into the capillary membrane 104. Buffer flowing from the conjugate pad 103 into the capillary membrane 104 after user actuation of the buffer reservoir 304 can then flow perpendicular to the buffer from the conjugate pad 103. The amount of sample flowing into the capillary membrane 104 can be defined by the intersecting surface area of the sample pad 102 and the capillary membrane 104.

FIG. 3C depicts an exploded view of the cassette 300 in the area around the buffer reservoir 305 and sample receptacle 306. With the upper housing 301 displaced from the lower housing 302, the exploded view shows placement of the LFIA test strip 100 relative to the sample preparation area. Additionally, the example gating device 306 is shown in greater detail.

In some embodiments, gating device 306 includes a button 306a in the shape of a post that is sized and shaped to protrude through a first corresponding hole/opening in the upper housing 301 when assembled. Button 306a is connected via a lateral member to the blocking post 306b (a sealing portion) that is sized and shaped to protrude through a second corresponding hole/opening 308 when assembled. The In particular, the blocking post 306b is sized and shaped to engage and form a seal with the hole to prevent fluid from escaping the sample receptacle 305 when the gating device 306 is in a first position. A distal end of the gating device 306 connects to a sidewall of the lower housing 302. The connection of the gating device 306 is hinged or otherwise arranged to permit some flex in the gating device 306. With this arrangement, a user can apply a downward force on the button 306a to flex the gating device 306, lower the button 306a, and cause a corresponding lowering of the blocking post 306b to a second position. As a result of the downward motion of the blocking post 306b, the seal is broken between blocking post 306b and hole 308 and the mixed reagent-sample solution can flow onto the test strip. The gating device 306 is generally biased so that absent external forces (e.g., a user's depression on button 306a), the blocking post 306b naturally forms the seal with hole 308. In some embodiments, a spring 309 is positioned beneath the button 306a or another portion of the gating device 306 to provide the biasing force. Other gating devices 306 are also contemplated to enable the selective release of fluid from the sample receptacle 305. For example, an adhesive (e.g., sealing strip) may be adhered to an underside of the hole 308, and a user may pull a tab at the end of the adhesive to remove the adhesive and permit flow of the fluid out of the sample receptacle 305. In particular, the mixed reagent-sample solution can flow from the sample pad into the conjugate pad 103, and along the capillary membrane 104 into one or more reporting areas. The mixed sample solution can then flow into the absorbent pad 105 for disposal after completion of the test.

FIGS. 3D-3I depict additional views of the cassette device 300.

Although various examples have been described in detail above, other modifications are possible. Accordingly, other implementations are within the scope of the following claims.

1. A cassette device for lateral flow immunoassay testing, comprising:
a housing;
a receptacle located on a top portion of the housing; and
a gating device, wherein:
the housing defines an interior space to accommodate a test strip,
the receptacle is arranged to hold a fluid above the test strip,
the gating device includes a sealing portion that selectively seals an opening defined in a base of the receptacle, and
the gating device is configured to be actuated between (i) a first position in which the sealing portion engages the opening in the base of the receptacle to form a seal and (ii) a second position in which the sealing portion disengages the opening in the base of the receptacle to break the seal.

2. The cassette device of claim 1, wherein, when the gating device is in the first position, the seal that is formed between the sealing portion of the gating device and the opening in the base of the receptacle is operable to substantially prevent a fluid from flowing out of the receptacle through the opening.

3. The cassette device of claim 1, wherein, when the gating device is in the second position and when the fluid is present in the receptacle, the fluid is capable of flowing through the opening in the base of the receptacle onto a portion of the test strip positioned beneath the receptacle.

4. The cassette device of claim 3, wherein the receptacle and the test strip are positioned with respect to each other such that the fluid flows through the opening onto a sample portion of the test strip.

5. The cassette device of claim 1, wherein the sealing portion of the gating device comprises a post having an outer diameter that corresponds to an inner diameter that enables the post to be slidably received through the opening in the base of the receptacle.

6. The cassette device of claim 1, wherein the housing further includes a second opening located a positive distance from the receptacle, and the gating device further includes a button portion that protrudes through the second opening such that, when the gating device is in the first position, the button portion is capable of being depressed by an external force to actuate the gating device into the second position.

7. The cassette device of claim 1, wherein the housing includes a viewing window that comprises an opening positioned above one or more reporting areas of the test strip.

8. The cassette device of claim 1, wherein the gating device is biased by default in the first position.

9. The cassette device of claim 8, further comprising a spring located in the interior space of the housing, wherein the spring is operable to facilitate biasing of the gating device in the first position.

10. The cassette device of claim 1, wherein the fluid comprises a mixture of a reagent and a biological sample collected from a test subject.

11. The cassette device of claim 10, wherein the biological sample comprises blood, serum, saliva, or urine.

12. The cassette device of claim 1, wherein the cassette device is configured for use in testing for COVID-19.

13. The cassette device of claim 1, wherein the cassette device is configured for use in testing for a bacterial infection, a viral infection, a cancer, a cholesterol level, or an antibody.

14. The cassette device of claim 1, further comprising:
a fluid reservoir located on the top portion of the housing adjacent to the receptacle; and
a channel between the fluid reservoir and the receptacle.

15. The cassette device of claim 14, wherein the fluid reservoir is arranged to hold a solution for mixing with a biological sample disposed in the receptacle.

16. The cassette device of claim 15, further comprising an actuator that, when actuated, causes a seal to break that (i) opens the channel to permit the solution to flow from the fluid reservoir to the receptacle or (ii) punctures a sack of the fluid reservoir that holds the solution to permit the solution to flow out of the sack and through the channel into the receptacle.

17. The cassette device of claim 16, wherein the actuator comprises a hinged lid having one or more protrusions.
